(19) United States
(12) Patent Application Publication
(10) Pub. No.: US 2005/0069972 A1
(43) Pub. Date: Mar. 31, 2005

(54) Kit for detecting endotoxin

(75) Inventors: Carlos A. Castro, Cotuit, MA (US); Richard J. Ridge, Falmouth, MA (US); Thomas J. Novitsky, East Falmouth, MA (US)

Correspondence Address: LOWRIE, LANDO & ANASTASI, RIVERFRONT OFFICE, ONE MAIN STREET, ELEVENTH FLOOR, CAMBRIDGE, MA, 02142, US

(73) Assignee: Associates of Cape Cod, Inc., East Falmouth, MA (US)

(21) Appl. No.: 10/897,979

(22) Filed: Jul. 23, 2004

Related U.S. Application Data
(63) Continuation-in-part of application No. 10/867,162, filed on Jun. 14, 2004.
Application No. 10/867,162 is a continuation-in-part of application No. 10/826,922, filed on Apr. 19, 2004.
(60) Provisional application No. 60/463,737, filed on Apr. 18, 2003.

(51) Int. Cl. C12Q 1/04; C12Q 1/34

(52) U.S. Cl. 435/34

(57) ABSTRACT

Kits and method for detecting bacterial endotoxin in an aqueous solution are provided. In certain examples, the kit includes at least a first container comprising solid, endotoxin-specific, horseshoe crab amebocyte lysate and at least one buffer, whereby the sensitivity of the amebocyte lysate is pre-certified. In certain examples, the kit also contains at least a second container comprising a defined quantity of endotoxin configured as a positive product control, wherein the defined quantity of the endotoxin is pre-certified to react positively with the amebocyte lysate in the first container.

### CROSS-REFERENCE TO RELATED APPLICATIONS

The application claims the priority benefit of and is a continuation-in-part of U.S. application Ser. No. 10/867,162 filed on Jun. 14, 2004, which was a continuation-in-part of U.S. application Ser. No. 10/826,922 filed on Apr. 19, 2004, which claimed priority to U.S. Provisional Application No. 60/463,737 filed on Apr. 18, 2003, the entire contents of each of which is hereby incorporated herein by reference for all purposes.

### FIELD OF THE TECHNOLOGY

Certain examples disclosed herein relate to methods and kits for detecting bacterial endotoxin. More particularly, certain examples disclosed herein relate to methods and kits for detecting bacterial endotoxin in aqueous solutions, such as water, dialysate, etc., using a Limulus Amebocyte Lysate (LAL)-based gel-clot assay.

### BACKGROUND

Bacterial endotoxins, also known as pyrogens, are the fever-producing byproducts of Gram-negative bacteria and can be dangerous or even deadly to humans. Symptoms of infection and presence of endotoxin range from fever, in mild cases, to death.

Cells from the hemolymph of the horseshoe crab (amebocytes) contain an endotoxin-binding protein (Factor C) that initiates a series of complex enzymatic reactions resulting in clot formation when the cells are in contact with endotoxin (reviewed in Iwanaga, Curr. Opin. Immunol. 5: 74-82 (1993)). The endotoxin-mediated activation of an extract of these cells, i.e., amebocyte lysate, is well-understood and has been thoroughly documented in the art. See, for example, Levin et al., Thromb. Diath. Haemorrh. 19: 186-197 (1968); Nakamura et al., Eur. J. Biochem. 154: 511-521 (1986); Muta et al., J. Biochem., 101: 1321-1330 (1987); and Ho et al., Biochem. Mol. Biol. Int., 29: 687-694 (1993). This phenomenon has been exploited in bioassays to detect endotoxin in a variety of test samples, including human and animal pharmaceuticals, biological products, research products, and medical devices.

The horseshoe crab Limulus polyphemus is particularly sensitive to endotoxin. Accordingly, the blood cells from this horseshoe crab, termed “Limulus amebocyte lysate” or “LAL”, are employed widely in endotoxin assays of choice because of their sensitivity, specificity, and relative ease for avoiding interference by other components that may be present in a sample. See, e.g., U.S. Pat. Nos. 4,495,294, 4,276,050, 4,273,557, 4,221,865, and 4,221,866. LAL, when combined with a sample containing bacterial endotoxin, reacts with the endotoxin to produce a product, for example, a gel clot or chromogenic product, that can be detected, for example, either visually, or by the use of an optical detector.

Although the enzymatic clotting cascade of LAL initially was considered specific for endotoxin, it was later discovered that β-(1,3)-D-glucans also activate the clotting cascade of LAL through a partially shared pathway, referred to as the Factor G pathway. See, for example, Morita et al., FEBS Lett. 129: 318-321 (1981); and Iwanaga et al., J. Protein Chem. 5: 255-268 (1986). Accordingly, if a sufficient amount of β-(1,3)-D-glucans are present in a sample, a LAL positive response may occur that is independent of the endotoxin-mediated response. Thus, it has become very important to increase the specificity of LAL for endotoxin, i.e., by utilizing an endotoxin specific amebocyte lysate preparation.

In one approach for achieving endotoxin-specificity of amebocyte lysate, polysaccharide based Factor G inhibitors are combined with amebocyte lysate to reduce or eliminate clotting induced by β-(1,3)-D-glucans present in the biological sample. See, for example, U.S. Pat. Nos. 5,155,032; 5,474,984; and 5,641,643.

Other approaches are known by those skilled in the art for increasing the specificity of LAL for endotoxins. For example, U.S. Pat. No. 5,401,647 discloses a method for removing Factor G from LAL by combining LAL with β-(1,3)-D-glucans immobilized on an insoluble carrier. Once bound to the carrier via the β-(1,3)-D-glucan moiety, Factor G can thereafter be removed from the LAL to produce a Factor G depleted lysate. Similarly, U.S. Pat. No. 5,605,806 discloses an immunoaffinity based method using a Factor G specific antibody to remove Factor G from LAL thereby to produce a Factor G depleted amebocyte lysate. Finally, Kakinuma, A. et al. describe a method that employs the addition of excess glucan to the lysate to overwhelm Factor G and prevent additional glucan from activating the lysate. See, Kakinuma, A. et al., Biochem. Biophys. Res. Commun. 101:434-439 (1981).

Endotoxins are a significant concern in the field of nephrology. About 300,000 individuals in the United States receive some form of dialysis, which provides life-saving renal replacement for end-stage renal disease (ESRD). Water for dialysis as well as dialysates is not sterile, and can contain significant concentrations of bacteria and endotoxins. Hemodialysis is a water-intensive therapy that presents an enormous challenge to produce copious amounts of high purity water, cost effectively.

In a typical dialysis system, blood and dialysate are pumped into the dialyzer (also known as the artificial kidney) from opposite directions. If the hydrostatic pressure on the dialysate side of the dialysis membrane exceeds the pressure on the blood side, it is possible to transfer endotoxins from the dialysate into the blood (back-filtration). In addition, endotoxins adsorbed to the membrane surface, resulting from a manufacturing error or deposited during a previous use, may be dislodged when the artificial kidney is initially primed with dialysate.

The occurrence of endotoxin-mediated pyrogenic reactions continues to challenge dialysis facilities. The potential for exposure of dialysis patients to greater levels of microbial and endotoxin contamination has increased dramatically during the last decade with the increase in re-use of hemodialyzers and the use of bicarbonate dialysate and high flux dialysis. See, Bland, L. A., Adv. Ren. Replace Ther. 2:70-79 (1995).

There are significant reasons to reduce the exposure of hemodialysis patients to endotoxins. The most acute is obviously to eliminate pyrogenic reactions. However, even more critical are the well-documented effects of long-term exposure to pyrogens, including leukocyte and monocyte activation, platelet activation, increased adhesiveness and aggregation, and complement activation, which together with hyperlipidemia, cause endothelium damage and lipid deposition in the arterial wall. Therefore, it is expected that regular use of sterile and endotoxin-free dialysate will help decrease the cardiovascular morbidity and mortality rate of patients undergoing hemodialysis. See, e.g., Amato, R. L., Nephrology Nursing Journal 28: 619-629 (2001).

Chronic inflammatory responses due to long-term consequences of cell stimulation and the subsequent release of inflammatory mediators such as tumor necrosis factor (TNF) and interleukin-1 (IL-1) are a major concern as well. See, Canaud, B., et al., “Microbiologic Purity of Dialysate: Rationale and Technical Aspects,” in Chronic Inflammation in Hemodialysis, pp. 34-47, Switzerland: S. Karger AG (2000). The use of sterile and endotoxin-free dialysate significantly decreases the interleukin levels in patients' blood.

Dialysis amyloidosis is considered an inflammatory disease; the major protein of amyloid deposits is beta-2-microglobulin. Synthesis of beta-2 microglobulin in macrophages is enhanced by endotoxins. Therefore, dialysis water contaminated with endotoxin may contribute to this process. Bad et al. showed that the onset of amyloidosis in long-term dialysis patients was considerably delayed when ultrapure dialysate was used (Bad, M. et al., Int. J. Artif. Organs 14:681-685 (1991)).

To help prevent pyrogenic reactions and bacteremia in hemodialysis patients caused by microbial and endotoxin contamination of hemodialysis fluids, the Association for the Advancement of Medical Instrumentation (AAMI) has recently approved standards for maximum allowable concentrations of bacteria and endotoxin in these fluids (endotoxin level should not exceed 2.0 EU/mL as tested by the LAL assay, and action must be taken when the level exceeds 1.0 EU/mL). It has been recommended that each dialysis center develop microbiological and endotoxin surveillance policies and procedures for the types of hemodialysis fluids to assay, frequency and manner of sample collection, assay techniques, and methods for recording and interpreting results to ensure compliance with the AAMI standards. Clearly, a safer environment would be provided for each dialysis patient if appropriate microbiological assay procedures are followed and the results are consistently within the AAMI microbiological and endotoxin standards.

Currently, available LAL tests on dialysate rely on one of three methods: The first is a standard gel-clot assay. This assay takes 60 minutes and requires the user to ‘select’ a sensitivity, which is unique to a particular lot of LAL. If the user wants to have control over the sensitivity or shorten the assay time (<60 minutes), they need to use one of the two photometric LAL methods, either the turbidimetric or the kinetic chromogenic method. Both of these methods, however, require specialized technical expertise and a machine to read the test, e.g., a microplate reader. Further, photometric LAL methods are expensive.

### SUMMARY

There is a need for rapid, simple, and cost-effective methods and kits for specifically detecting endotoxin in aqueous solutions such as water and dialysate solutions that would combine the ease of use of a gel-clot LAL assay with the speed and multi-sensitivity of the photometric methods, but without requiring specialized equipment or expertise. This need is particularly felt in the renal dialysis clinic.

Certain aspects and examples disclosed herein provide simple methods and kits for specifically detecting endotoxin. More particularly, certain aspects and examples disclosed herein provide rapid and cost-effective methods and kits for specifically detecting endotoxin in aqueous solutions, such as water or dialysate solutions. Certain examples of the methods and kits disclosed herein combine the ease of use of a gel-clot assay with the speed and variable sensitivity of a photometric method without the use of specialized equipment or expertise. These and other aspects, examples and advantages are discussed in more detail below.

In accordance with a first aspect, a kit for specifically detecting endotoxin is provided. In certain examples, the kit comprises at least a first container containing solid, endotoxin specific, horseshoe crab amebocyte lysate and at least one buffer, whereby the sensitivity of the lysate is pre-certified. In certain examples, the kit also comprises a second container containing a defined quantity of endotoxin configured to serve as a positive product control (PPC), wherein the defined quantity of endotoxin is pre-certified to react positively with the amebocyte lysate present in the first container. In some examples and as discussed further below, the control in the second container is a “matched” PPC sample of endotoxin. In certain other examples, the kit may also comprise at least one disposable endotoxin free transfer instrument. In some examples, the sensitivity of the kit (i.e., the amount of endotoxin (EU) the kit can detect) can vary based on numerous factors including, but not limited to, the time of incubation of the test, and the formulation of the lysate in the first container. Also, the ability of the kit to indicate the absence of sample interference or inhibition can be based, at least in part, on the quantity of endotoxin contained in the PPC.

In accordance with another aspect, methods and kits for use in kidney dialysis clinics and kidney dialysis procedures are disclosed. In certain examples, the LAL assay described herein is especially useful in the kidney dialysis clinic. During kidney dialysis, blood is circulated through a machine which contains a dialyzer. The dialyzer has two spaces separated by a thin membrane. Blood passes on one side of the membrane and dialysis fluid passes on the other. The wastes and excess water pass from the blood through the membrane into the dialysis fluid which is then discarded. The cleansed blood is returned to the patient's bloodstream.

In accordance with an additional aspect, methods and kits for testing water systems are provided. In certain examples, the endotoxin-specific LAL kit described herein may be used to routinely and more frequently test water systems used to prepare dialysate, flush lines, and prime dialysis machines prior to use by each patient. The LAL kit described herein may also be used to test the salt solutions used throughout the actual dialysis session.

In accordance with another aspect, endotoxin-specific horseshoe crab amebocyte lysate is provided. In certain examples, the endotoxin-specific horseshoe crab amebocyte lysate in the first container is isolated from Limulus polyphemus. Although certain examples described below refer to Limulus amebocyte lysate (LAL), other suitable horseshoe crab amebocyte lysates will be readily selected by the person of ordinary skill in the art, given the benefit of this disclosure.

In accordance with an additional aspect, endotoxin-specific horseshoe crab amebocyte lysate is disclosed. In certain examples, the horseshoe crab amebocyte lysate is made endotoxin-specific by using horseshoe crab amebocyte lysate factor G activation inhibitor in accordance with the teachings in U.S. Pat. Nos. 5,641,643, 5,474,984, or 5,155,032, for example. Each of these patents is incorporated herein by reference in their entirety for all purposes. In certain examples, the amebocyte lysate is solid, e.g., is a freeze-dried, salted-out or precipitated lysate.

In accordance with another aspect, the amounts and sensitivities of the endotoxin-specific LAL reagent can vary, and exemplary amounts and sensitivities are discussed below. In certain examples, the amebocyte lysate is in lyophilized form and will be reconstituted during the assay, e.g., reconstituted with sample. The sensitivity of the LAL reagent is pre-certified against the United States Pharmacopeia endotoxin standard. In certain examples, the second container comprises a “matched” PPC sample of endotoxin. Without wishing to be bound by any particular scientific theory, to understand better the concept of a “matched” PPC, reference is made below to the endotoxin PPC in a conventional LAL test. In conventional LAL tests, an endotoxin positive PPC is typically prepared by diluting a concentrated endotoxin standard to an appropriate concentration, so that when the standard is added to the sample, a concentration of 2× the sensitivity of the LAL being used results, i.e., 2× lambda results where lambda is the sensitivity of the LAL. In the conventional test, the addition of the endotoxin to the sample to make the PPC results in a slight dilution, which can adversely affect the outcome of the test. In addition to the dilution effect, the preparation of this PPC can be extremely variable and depends on the skill of the user and the quality of the accessories, i.e., diluents, tubes, pipettes, etc. The “matched” control provided herein comprises a defined quantity of endotoxin (Endotoxin Units, EU) that is pre-certified to be about 2× lambda or about twice the sensitivity of the lysate, thereby ensuring a positive reaction with the LAL contained in the first test tube. In some examples, the matched control is exactly 2× lambda or exactly twice the sensitivity of the lysate. The term “matched PPC” refers to the defined amount of endotoxin standard in the second container (i.e., the PPC) that has been previously tested and has been certified to be about 2× lambda to provide a positive result when combined with the LAL component of the first container. Using a pre-certified, matched PPC provides numerous advantages including, for example, a high degree of assurance of a valid test, i.e., a test that is not inhibited by the test sample, simpler and more rapid assays, etc. In addition, when using the methods and kits disclosed herein, the user does not need to run a standard or a negative control since all the components of the assay have been certified. For example, a Certificate of Compliance attesting to the amebocyte lysate sensitivity, endotoxin concentration of the PPC, and/or the endotoxin-free nature of the transfer instrument may be provided with each kit.

In accordance with other aspects, the first and second containers may take numerous forms. In certain examples, the first and second container(s) in the kit are independently selected from vials, test tubes, centrifuge tubes, flasks, Eppendorf tubes, microcentrifuge tubes, U-shaped tubes, blood collection tubes, thistle tubes, hybridization tubes, capillary tubes, wintrobe tubes, culture tubes, microtiter tubes, hematocrit and microhematocrit tubes, and the like. In certain examples, the containers are each test tubes that are 12 mm×75 mm and are round-bottomed.

In accordance with another aspect, the containers in the kit and/or any caps of sealing devices including with the containers may be color-coded and identified by ink-jet or other suitable labels (e.g., “sample”; “Control”) on the tubes themselves to prevent sample mix-ups. Other suitable methods and devices to provide easier handling of the kit components will be readily selected by the person of ordinary skill in the art, given the benefit of this disclosure.

In accordance with yet an additional aspect, the disposable endotoxin-free transfer instruments that can optionally be included in the kit may take various sizes and forms. In certain examples, the transfer instrument is a disposable pipette, a transfer pipette, a volumetric pipette, a syringe, a capillary tube, disposable pipette and/or micropipette tips such as those commonly used with automatic pipetters, and other suitable devices that can be made endotoxin-free.

In accordance with another aspect, kits comprising a first and second container may further include written instructions for the user or refer the user to protocols or methods that have been adopted by an organization, such as AAMI, for example.

It will be recognized by the person of ordinary skill in the art, given the benefit of this disclosure, that the methods and kits disclosed herein provide simple and rapid assays that can be used to detect bacterial endotoxin with high specificity, precision and accuracy and with minimal or no interferences. Robust kits can be provided to detect bacterial endotoxin in water, dialysate or other liquids and/or solutions where it may be necessary to determine the presence of endotoxin. These and other advantages and uses of the methods and kits disclosed herein are described in more detail below.

### BRIEF DESCRIPTION OF THE FIGURES

Certain examples are described below with reference to the accompanying figures in which:

FIG. 1 is an example of a first container comprising solid LAL and buffer and a second container comprising a matched PPC, in accordance with certain examples;

FIG. 2 is an example of a first container comprising an aqueous solution added to lyophilized LAL, and a second container comprising a matched PPC, in accordance with certain examples;

FIG. 3 is an example of transferring one-half of the solution from the first container to the second container, in accordance with certain examples; and

FIG. 4 is an example of first and second containers ready for incubations, in accordance with certain examples.

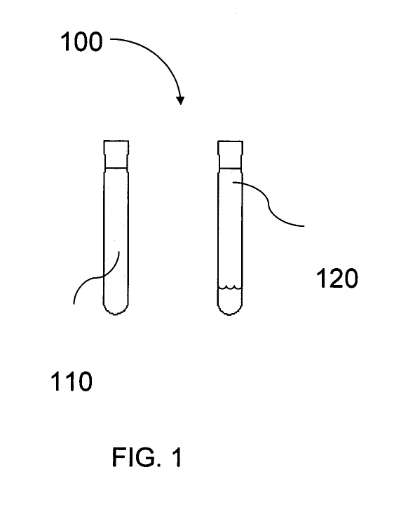

The containers shown in FIGS. 1-4 are for illustrative purposes only, and the exact size of the containers can vary depending on the nature of the container, sample size, etc.

### DETAILED DESCRIPTION OF CERTAIN EXAMPLES

Certain examples disclosed below describe the use of endotoxin-specific LAL and a matched PPC for detection of endotoxin in aqueous solutions, such as water or dialysate solutions. It will be within the ability of the person of ordinary skill in the art, given the benefit of this disclosure, however, to detect endotoxin in these and other solutions using the methods and kits disclosed herein. The LAL assay described herein is particularly useful in the dialysis clinic. The assay may be used to routinely, and thus more frequently, test the water systems used to prepare dialysate, flush lines, and prime dialysis machines prior to use by patients. The LAL assay described herein may also be used to test the salt solutions (dialysate) used in the actual dialysis machine.

As used herein, the term “aqueous solution” refers to any sample of purified, distilled, sterile, non-sterile, or filtered water, water for injection, water for irrigation, or reverse osmosis water, or any aqueous solution used in connection with hemodialysis, peritoneal renal dialysis, pre-operative organ perfusion, and/or organ (e.g., renal) transplantation, in which it would be useful to determine possible endotoxin contamination. The term “dialysate” is a particular example of such an aqueous solution and is intended to refer to the salt solutions used in the dialysis process. Dialysates can occasionally inhibit an ordinary LAL test (e.g., give false negative). Certain examples disclosed herein are designed to overcome inhibition or false negative reactions with all commonly used dialysates. Other aqueous solutions that may be tested, include, e.g., saline and other salt solutions, as well as solutions of sugar, such as dextrose water.

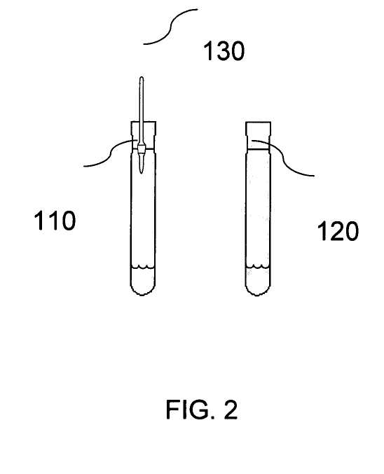

In accordance with certain examples, a kit for assaying endotoxin is provided. Referring now to FIG. 1, kit 100 includes first container 110 and second container 120. In certain examples, first container 110 comprises solid, e.g., freeze dried, endotoxin-specific, horseshoe crab amebocyte lysate and at least one buffer, whereby the sensitivity of the lysate is pre-certified. In certain examples, second container 120 comprises a defined quantity of endotoxin configured to serve as a PPC, wherein said defined quantity of endotoxin is pre-certified to react positively with the amebocyte lysate present in the first container. In certain examples, the kit also includes at least one disposable endotoxin-free transfer instrument, such as transfer instrument 130 shown in FIG. 2. Without wishing to be bound by any particular scientific theory, the sensitivity of the kit can vary based on the time of incubation of the two containers in the kit. Also, the validity of the kit is based, at least in part, on the quantity of endotoxin contained in the PPC.

In accordance with certain examples, the endotoxin-specific horseshoe crab amebocyte lysate used in the first container may be isolated from any of the four known species of horseshoe crab: Limulus polyphemus, Tachypleus gigas, Tachypleus trident-atus or Carcinoscorpius rotundicauda. Particularly useful lysate is amebocyte lysate isolated from Limulus polyphemus, the horseshoe crab found along the North American coast. Although the Limulus amebocyte lysate (LAL) is particularly useful and may be specifically cited when describing other components herein, it is emphasized that other horseshoe crab amebocyte lysates will be readily selected by the person of ordinary skill in the art, given the benefit of this disclosure. In at least certain examples, the amebocyte lysate is in lyophilized form and will be reconstituted during the assay. For example, LAL with enhanced sensitivity to endotoxin can be prepared according to the teachings in U.S. Pat. No. 4,107,077 and utilized as the reagent in the first container. This patent is hereby incorporated by reference herein in its entirety for all purposes.

In accordance with certain examples, specific LAL formulations, comprising particular combinations and types of buffers can be used. These specific LAL formulations can impart functionality to the lysate by overcoming inhibition that may be encountered when testing dialysate and other salt solutions. Certain specific LAL formulations are discussed in more detail below. The exact nature of the buffer can vary depending on the desired pH range. For example, the buffer may be selected from one or more of imidazole, Tris, HEPES, histidine, Bis-Tris, MES, citrate and other suitable buffers that can resist changes in pH in the pH 6-8 range. In some examples, the buffer is co-lyophilized with the LAL reagent, whereas in other examples, the buffer is added to lyophilized LAL in a suitable amount to provide a desired buffering capacity and/or a desired pH. The exact concentration of the buffer can vary, for example, depending on the pH of the sample to be tested. In examples where an aqueous sample, e.g., water or dialysate, around is tested, little buffering may be needed so low amounts of buffer can be used, e.g., about 10 mM or less buffer can be used. In examples where the sample to be tested is at a lower pH (<7) or a higher pH (>7), it may be necessary to use higher buffer concentrations, e.g., about 10 mM to about 0.1 M buffer. In certain examples, the pH of the buffer is adjusted to about 6-8, more particularly about 7, using one or more suitable acids or bases, such as HCl or NaOH, respectively, for example. It will be within the ability of the person of ordinary skill in the art, given the benefit of this disclosure, to select suitable buffers and suitable amounts of buffers for use in the kits and methods disclosed herein. In some examples where the buffer is added to the lysate prior to lyophilization, an imidazole buffer is used and is present at about 0.010-0.1 M, e.g., about 0.025 M, and pH 7 prior to lyophilization.

In accordance with certain examples, the LAL may also include one or more salts, such as metal or non-metal salts to enhance functionality of the lysate. In certain examples, one or more metal or non-metal sulfate, sulfite, chloride, bromide, iodide, cyanate, isocyanate, thiocyanate, chromate, phosphate, carbonate, nitrate, chlorite, acetate, etc. can be used in the LAL formulation. In certain examples, one or more of magnesium or sodium salts are used such as, for example, magnesium sulfate and sodium chloride. The exact concentration of the salt can vary, and in certain examples, the concentration of the salt is about 0.01-1.0 M or about 0.1% to about 4% by weight. When a magnesium sulfate salt is used, the concentration is typically around 0.02-0.1 M, more particularly about 0.05 M. When a sodium chloride salt is used, the concentration is typically around 1% to about 3% by weight, more particularly about 1% by weight.

In accordance with certain examples, horseshoe crab amebocyte lysate can be made endotoxin-specific by using the horseshoe crab amebocyte lysate factor G activation inhibitor in accordance with the teachings in U.S. Pat. Nos. 5,641,643, 5,474,984, or 5,155,032. Each of these patents is hereby incorporated by reference herein in its entirety for all purposes. Other suitable techniques of making the amebocyte lysate endotoxin-specific will be readily selected by the person of ordinary skill in the art, given the benefit of this disclosure. As used herein, the term “endotoxin-specific” refers to the amebocyte lysate in the first container of the kit that does not substantially or appreciably react with substances, e.g., β-(1,3)-D-glucans, other than bacterial endotoxin and cause a false positive result.

In accordance with certain examples, the first and second container may take numerous forms. For example, in certain configurations of the kit, the first and second containers each is independently selected from vials, test tubes, centrifuge tubes, flasks, Eppendorf tubes, microcentrifuge tubes, U-shaped tubes, blood collection tubes, thistle tubes, hybridization tubes, capillary tubes, wintrobe tubes, culture tubes, microtiter tubes, hematocrit and microhematocrit tubes and the like. In certain other examples, each of the first and second container is a 12 mm×75 mm test tube, and more particularly, a round bottom 12 mm×75 mm test tube. The test tubes in the kit and/or any test tube caps may be color-coded and identified by ink-jet or other suitable labels (e.g., “Sample”; “Control”) on the tubes themselves to prevent sample mix-ups.

In accordance with certain examples, the first container comprises an endotoxin-specific LAL reagent in solid form, e.g., in lyophilized form. In certain examples, the total volume of LAL reagent is about 0.5 mL and provides sufficient volume to account for any pipetting loss during transfer from the first container to the second container. The sensitivity of the LAL reagent is pre-certified against the United States Pharmacopeia endotoxin standard be selected based on the desired sensitivity. For example, in certain applications, the amount is selected to provide a sensitivity of about 2.0 EU/mL or less, more particularly about 1 EU/mL or less, for example about 0.5 EU/mL or less, about 0.25 EU/mL or less, about 0.125 EU/mL or less, about 0.03 EU/mL or less, or about 0.015 EU/mL or less. As discussed in more detail below, the LAL reagent is reconstituted with sample to provide a total volume of about 0.3-0.7 mL, e.g., 0.4 mL-0.6 mL total volume, and in certain examples, 0.5 mL total volume is used.

In accordance with certain examples, the second container comprises a matched PPC of endotoxin. To provide a more user-friendly description of the concept of a “matched” PPC, reference is made below to endotoxin PPC in a conventional LAL test. In conventional LAL tests, an endotoxin PPC is typically prepared by diluting a concentrated endotoxin standard to an appropriate concentration, so that when the standard is added to the sample, a concentration of 2× the sensitivity of the LAL being used (i.e., 2× lambda) results, where lambda is the sensitivity of the LAL. In the conventional LAL test, the addition of the endotoxin to the sample to make the PPC results in a slight dilution, which can adversely affect the outcome of the test. In addition to the dilution effect, the preparation of this PPC can be extremely variable and depends on the skill of the user and the quality of the accessories, i.e., diluents, tubes, pipettes, etc. The “matched” PPC provided herein in the second test container comprises a defined quantity of endotoxin (Endotoxin Units, EU) that is pre-certified to be about 2× lambda, or exactly 2× lambda, or about twice the sensitivity of the lysate, thereby ensuring a positive reaction with the LAL contained in the first test tube. Thus, the term “matched PPC” refers to the defined amount of endotoxin standard in the second container (i.e., the PPC) that has been previously tested and certified to be about 2× lambda and to give a positive result when combined with the LAL component of the first container. Using a pre-certified, matched PPC provides a high degree of assurance of a valid test, i.e., a test that is not inhibited by the test sample. Furthermore, using the methods and kits disclosed herein, the user of the kit does not have to run a standard or a negative control since all the components of the kit have been certified. The kit may also further comprise a Certificate of Compliance of the amebocyte lysate sensitivity, the endotoxin concentration of the PPC, and/or the endotoxin-free nature of the transfer instrument (e.g., the pipette). In certain examples, the kit may further include user instructions and any additional information that may be helpful to a user. It will be within the ability of the person of ordinary skill in the art, given the benefit of this disclosure, to include suitable instructions and certificates in the kits disclosed herein.

In accordance with certain examples, the disposable endotoxin-free transfer instruments, which can optionally be included in the kit, are transfer pipettes. Of course, other pipetting devices known to those skilled in the art, e.g., glass or plastic, graduated or volumetric, pipettes and mechanical pipetters with removal tips may be used, so long as they are substantially endotoxin-free. Endotoxin-free syringes may also be used to transfer solutions, most typically, through the sealed cap of the container. Additional suitable transfer devices will be readily selected by the person of ordinary skill in the art, given the benefit of this disclosure.

In accordance with certain examples, several conventional methods are used to test dialysate for endotoxin. A first method is a standard gel-clot assay. The gel-clot assay takes about 60 minutes and requires the user to “select” a sensitivity, which is unique to a particular lot of LAL. If a user wants control over the sensitivity or shorten the assay time (<60 minutes), they need to use one of the photometric LAL methods, either turbidimetric or chromogenic. However, both of these methods require higher skill to use and also an instrument to read the test, e.g., a microplate reader. The methods and kits disclosed herein combine the ease of use of the gel-clot assay with the speed and variable sensitivity of the photometric methods without the use of specialized equipment or expertise.

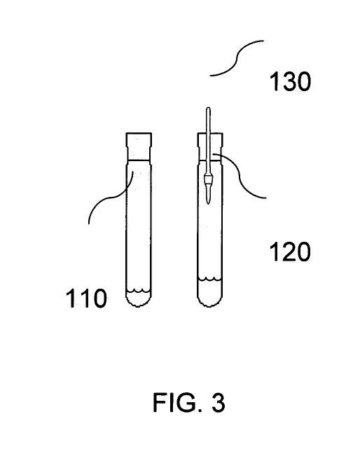

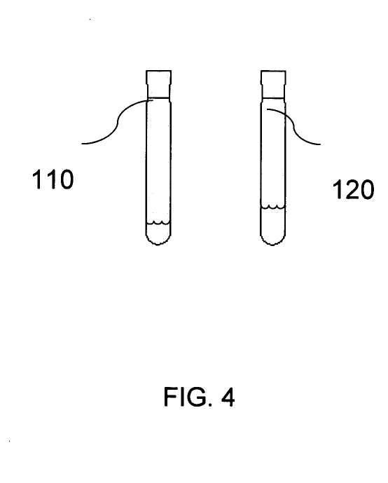

In accordance with certain examples and referring to FIG. 1, a user adds a suitable volume, e.g., 0.5 mL, of sample directly to first container 110 which comprises LAL reagent. Unlike standard LAL tests, there is no need to first reconstitute the LAL with endotoxin-free water prior to sample addition. The assays described herein use the sample to reconstitute the LAL reagent. The sample is added to first container 110 using transfer instrument 130 (see FIG. 2), which can optionally be provided with the kit. The LAL reagent is reconstituted by swirling the first container with added sample for about 30-60 seconds. One-half of the volume in the first container, e.g., about 250 uL where 500 uL of sample is added to the first container, is removed using transfer instrument 130 and added to second container 120. See FIG. 3. Second container 120 with the added volume from the first container serves as a “matched” PPC. See FIG. 4. Using currently available FDA-approved single test vials, it is not possible to internally control the LAL test with a matched PPC since two separate and different LAL tubes are needed—one for the test and one for the control. Examples of the kit described herein provide a first container with twice the volume of LAL, which can be split and used in the matched PPC, thereby eliminating tube-to-tube variation, pipetting errors, etc. The kits and methods described herein provide a more accurate result overall. After swirling or tapping the second container, both containers are placed in a simple block heater or water bath at 37° C.±1° C. and incubated for a period of time dictated by the level of sensitivity desired. Such time periods may be 10, 20, 30, 40, 50, 60, 70, 80, 90, 100, 110, or 120 minutes, or any time in between. The endotoxin content of the PPC is selected according to the sensitivity desired for the kit. Without wishing to be bound by any particular scientific theory, by using higher amounts of endotoxin, shorter incubation times can be used. For example, the incubation time may vary from about 15 minutes when using high concentrations of PPC (e.g., 1.0 EU/mL), to about two hours when using low concentrations of PPC (e.g., 0.005 EU/mL). One of the advantages of the methods and kits disclosed herein is the variable sensitivity of the assay. For example, if the sensitivity of the “sample” LAL is about 0.25 EU/mL (referred to as lambda), then the matched “Control” would contain about 2× lambda or 0.5 EU/mL. At this sensitivity, the test would be completed in less than 30 minutes, more particularly; the test would be completed in about 25 minutes. If positive “Controls” containing higher amounts of endotoxin are used, however, the test time will be even shorter; if controls containing lesser amounts of endotoxin are used, the test time will be longer. The sensitivity of the assay may be chosen by the user, and can range from about 2.0 EU/mL, 1.0 EU/mL, 0.5 EU/mL, 0.25 EU/mL, 0.125 EU/mL, 0.03 EU/mL, or 0.005 EU/mL of endotoxin (or any amount in between) depending on the time of the test. The timing of the test may vary from approximately 15 minutes when using high concentrations of PPC (e.g., about 1.0 EU/mL), to about two hours when using lower concentrations of PPC (e.g., about 0.005 EU/ml).

Certain illustrative specific examples are described below are not intended to limit the scope of the methods and kits described herein.

### EXAMPLE 1

### Formulation for the Horseshoe Crab Amebocyte Lysate Reagent in the First Container of the Kit

Amebocyte lysate, derived from Limulus polyphemus, is obtained using the methods described in U.S. Pat. No. 4,107,077. Amebocyte lysate may be stored frozen in aliquots both for convenience and preservation of activity. Prior to lyophilization and storage, the amebocyte lysate is formulated with other components at various final concentrations of 0.02-0.1 M MgSO₄, 1-3% NaCl, 0.01-0.1 M Imidazole-HCl buffer at pH 7, and 0.1-0.15 mg/mL Factor G Activation Inhibitor (described in U.S. Pat. No. 5,641,634).

### EXAMPLE 2

### Specific Formulation for the Horseshoe Crab Amebocyte Lysate Reagent in the First Container of the Kit

Amebocyte lysate, derived from Limulus polyphemus, was obtained using the methods described in U.S. Pat. No. 4,107,077. Amebocyte lysate was stored frozen at −80° C. in 250-1L aliquots both for convenience and preservation of activity. Prior to lyophilization and storage, the amebocyte lysate was formulated with other components at final concentrations of 0.05 M MgSO₄, 1% NaCl, 0.025 M Imidazole-HCl buffer at pH 7 and 0.125 mg/mL Factor G Activation Inhibitor.

### EXAMPLE 3

### Exemplary Kit Instructions for Detecting Endotoxin in an Aqueous Sample

1. An endotoxin-free pipette was used to add 0.5 mL of sample to a 12 mm×75 mm round bottom test tube (the “SAMPLE” tube) containing LAL reagent (as described in Example 2). The SAMPLE tube contained a suitable amount of LAL reagent to provide a sensitivity of about 0.25 EU/mL. Random samples of the pipettes were tested to ensure that endotoxin concentration present is less than 0.03125 EU/mL when delivered from the manufacturer (Sarstedt).
2. The contents of the tube were mixed by tapping the bottom of the tube lightly several times with a finger. The total mixing time was about 60 seconds.
3. Using the same pipette used in step #1 above, 0.25 mL of fluid in the first container was removed and transferred to a second 12 mm×75 mm test tube containing about 2 lambda quantity of endotoxin to serve as the PPC tube (the “CONTROL” tube).
4. The contents of the CONTROL tube were mixed by lightly tapping the bottom of the tube several times with a finger. The total mixing time was about 10 seconds.
5. After mixing the CONTROL tube, both the SAMPLE tube and the CONTROL tube were immediately placed in a 37° C. water bath.
6. Timing was started as soon as the tubes were placed in the incubator. In this example, an incubation time of 25 minutes was used.
7. After 25 minutes, the tubes were immediately and carefully removed one by one from the incubator. The tubes were gently inverted until the absence of a solid gel-clot was confirmed or to 180 degrees, i.e., complete, inversion was reached. If a solid clot had formed, the test result was positive for endotoxin. If no clot had formed (i.e., the mixture remained liquid, or the clot broke), the test result was negative for endotoxin. Using this assay, a test was considered to be valid and positive if the tube labeled “SAMPLE” was positive and the tube labeled “CONTROL” was also positive, i.e., both had solid gel-clots. A valid and positive result meant the sample contained greater than or equal to 0.25 EU/mL endotoxin.
8. A test was considered valid and negative if the tube labeled “SAMPLE” was negative and the tube labeled “CONTROL” was positive. A valid and negative result meant the sample contained less than 0.25 EU/mL.
9. A test was considered invalid if the tube labeled “CONTROL” was negative (no gel-clot) regardless of the results for the tube labeled “SAMPLE.” If this result occurred, the technique was checked and the test was repeated. If on repeating, the test was still invalid, the sample undergoing testing was deemed incompatible.

Although certain examples have been described above for purposes of clarity of understanding, the technology provided herein is not limited to the particular examples disclosed, but is intended to cover all alterations, substitutions, additions and modifications that are within the spirit and scope of the methods and kits as defined by the appended claims.

All publications and patents referred to in this disclosure are incorporated herein by reference to the same extent as if each individual publication or patent application were specifically and individually indicated to be incorporated by reference. Should the meaning of the terms in any of the patents or publications incorporated by reference conflict with the meaning of the terms used in this disclosure, the meaning of the terms in this disclosure are intended to be controlling.

1. A kit for detecting bacterial endotoxin in an aqueous solution, the kit comprising:
at least a first container comprising solid, endotoxin-specific, horseshoe crab amebocyte lysate and at least one buffer, whereby the sensitivity of the amebocyte lysate is pre-certified; and
at least a second container comprising a defined quantity of endotoxin configured as a positive product control, wherein the defined quantity of the endotoxin is pre-certified to react positively with the amebocyte lysate in the first container.

2. The kit of claim 1, in which the horseshoe crab amebocyte lysate is from Limulus polyphemus.

3. The kit of claim 1, in which the defined quantity of endotoxin in the second container is about two times the sensitivity of the amebocyte lysate in the first container.

4. The kit of claim 1, in which the horseshoe crab amebocyte lysate is present in a suitable amount to provide a sensitivity of about 1.0 EU/mL.

5. The kit of claim 1, in which the horseshoe crab amebocyte lysate is present in a suitable amount to provide a sensitivity of about 0.5 EU/mL.

6. The kit of claim 1, in which the horseshoe crab amebocyte lysate is present in a suitable amount to provide a sensitivity of about 0.125 EU/mL.

7. The kit of claim 1, in which the horseshoe crab amebocyte lysate is present in a suitable amount to provide a sensitivity of about 0.03 EU/mL.

8. The kit of claim 1, in which the aqueous solution is dialysate.

9. The kit of claim 1, in which the aqueous solution is purified water, distilled water, sterile water, non-sterile water, filtered water, water for injection, water for irrigation or reverse osmosis water.

10. The kit of claim 1, further comprising at least one endotoxin-free transfer instrument.

11. The kit of claim 1, in which the first and second container each is a test tube.

12. The kit of claim 1, in which the first and second container are independently selected from the group consisting of vials, centrifuge tubes, flasks, Eppendorf tubes, microcentrifuge tubes, U-shaped tubes, blood collection tubes, thistle tubes, hybridization tubes, capillary tubes, wintrobe tubes, culture tubes, microtiter tubes, hematocrit tubes and microhematocrit tubes.

13. The kit of claim 1, in which the solid, endotoxin-specific, horseshoe crab amebocyte lysate is freeze-dried, endotoxin-specific, horseshoe crab amebocyte lysate.

14. The kit of claim 1, in which the buffer is selected from the group consisting of imidazole, Tris, HEPES, histidine, Bis-Tris, MES, and citrate.

15. The kit of claim 1, in which the buffer is present at about 0.025 M and pH 7 in horseshoe crab amebocyte lysate prior to lyophilization.
